(19) United States
(12) Patent Application Publication
(10) Pub. No.: US 2020/0346035 A1
(43) Pub. Date: Nov. 5, 2020

(54) THERAPEUTIC RADIATION METHOD AND SYSTEM FOR TREATING ADDICTION

(71) Applicant: Regain Biotech Corp., New Taipei (TW)

(72) Inventors: LIANG-HSIANG WU, New Taipei (TW); NGOT-SWAN CHONG, New Taipei (TW); CHIH-CHING CHANG, New Taipei (TW); YI-CHING LIU, New Taipei (TW); MING-HSUN HSU, New Taipei (TW)

(21) Appl. No.: 16/677,225

(22) Filed: Nov. 7, 2019

(30) Foreign Application Priority Data
May 5, 2019 (TW) 108115479

(51) Int. Cl. A61N 5/10

(52) U.S. Cl. CPC A61N 5/1038; A61N 2005/1098; A61N 5/1039

(57) ABSTRACT

A method for treating addiction by therapeutic radiation including generating an original therapeutic plan according to a basic radiation dose; emitting radiation beams to a target area of an addict according to the original therapeutic plan, the target area comprising nucleus accumbens, the radiation beams based on the basic radiation dose capable of lowering an activity of the nucleus accumbens; and imaging the target area to detect whether the nucleus accumbens recover activity in a preset duration after the original therapeutic plan is completed. A detection of whether the nucleus accumbens recover activity is configured for determining a target radiation dose.

### FIELD

The disclosure relates to radiation therapy.

### BACKGROUND

Addictive substances, such as drugs and alcohol, can cause structural and functional changes of neurons in the brain. Such changes may last for a number of weeks, months, or even years, and weaken the effect of the addictive substance, thus leading to stronger addiction and a craving for greater quantities of the addictive substance. The brain's reward system plays a key role in the process of addiction, and has a close relationship with the mesolimbic dopamine system. The mesolimbic dopamine system includes a group of neurons positioned at the ventral tegmental area (VTA) near the base of the brain. Dopamine is produced by neurons in the VTA, while the nerve endings of the neurons extend to the prefrontal cortex and the nucleus accumbens. The transporting of dopamine from the VTA to the nucleus accumbens and the release of dopamine in the nucleus accumbens may result in addiction.

Existing treatments of addiction is mainly based on drugs and surgery. Treatment by drugs may lead to side effects such as nausea and vomiting, dizziness, lethargy, headache, dry mouth, sweating (mostly at night), confusion of consciousness, respiratory depression (especially when another central nervous system inhibitor is used at the same time), hypotension, dehydration, bile duct and renal tube convulsion, lack of appetite, nausea and vomiting, and menstrual changes. Furthermore, the effect of treatment by drugs only lasts for a short time, so addiction may not have overcome. Surgical treatment, taking stereotactic surgery for example, involves destruction of certain structures deep within the brain to block the transmission of nerve signals, thus reducing the desire for using the addictive substances. The main target during the stereotactic surgery is cingulate gyms or nucleus accumbens. Destruction of the cingulate gyms can break the thoughts and cravings of the substances being consumed. Destruction of the nucleus accumbens can block the transmission path of dopamine at the central cortical margin. Although the stereotactic surgery is useful, unpredictable side effects can arise, particularly important if the treatment is irreversible. Other surgical methods, such as deep brain stimulation (DBS) which implants electrodes stimulates the neurons with an external stimulator. DBS can cause less damage to brain tissues and is reversible. Stimulation by a voltage during DBS is adjustable, but DBS is still in the research stage, and the implanted electrodes in the brain may cause problems in a patient's lifestyle, making it difficult to be widely used in clinical practice.

### BRIEF DESCRIPTION OF THE DRAWINGS

Implementations of the disclosure will now be described, by way of embodiments only, with reference to the attached figures.

FIG. 1 is a block diagram of a therapeutic radiation system for treating addiction according to the present disclosure.

FIG. 2 is a diagrammatic view of a therapeutic radiation device of the therapeutic radiation system of FIG. 1.

FIG. 3 is a flowchart of a therapeutic radiation method for treating addiction according to the present disclosure.

FIG. 4 is a sub-flowchart of blocks 3 and 4 of FIG. 3.

### DETAILED DESCRIPTION

It will be appreciated that for simplicity and clarity of illustration, where appropriate, reference numerals have been repeated among the different figures to indicate corresponding or analogous components. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

FIG. 1 illustrate an embodiment of a therapeutic radiation system 100 for treating addiction of an addict 200. Addiction is a dependence on a substance or behavior that the addict 200 is unable to control. Substance addictions can include for example alcoholism, drug abuse, and smoking. Behavior addictions can include for example, gambling, overshopping, and overeating.

The system 100 includes a plan generator 10, a therapeutic radiation device 20, and an imaging device 30.

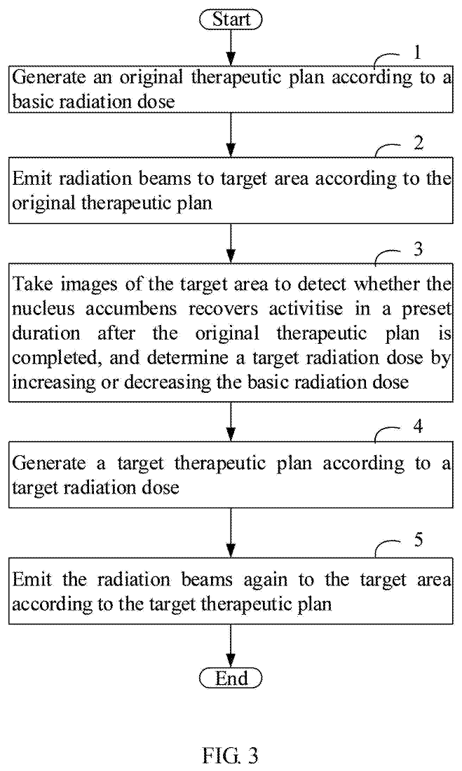

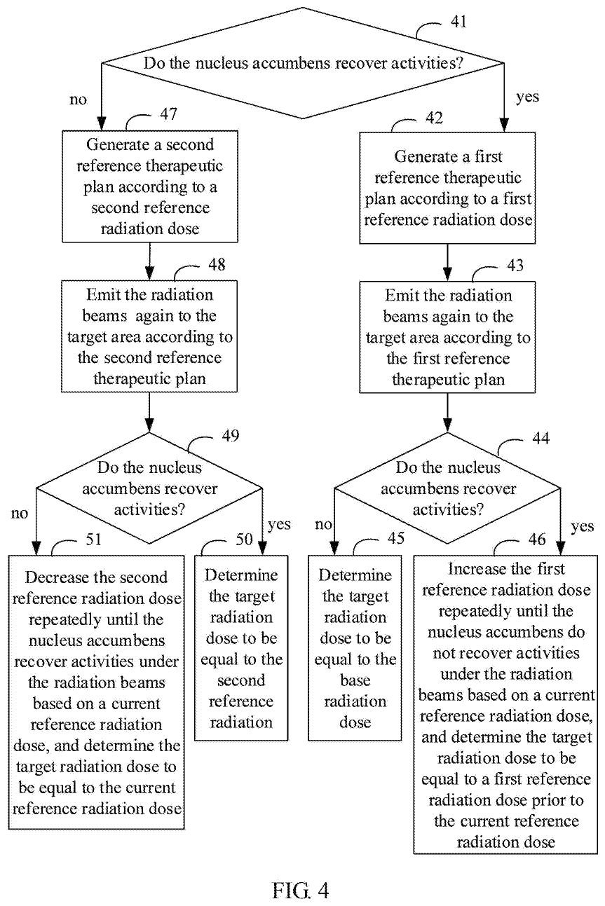

FIG. 3 illustrates an embodiment of a therapeutic radiation method for treating addiction. The method is provided by way of embodiments, as there are a variety of ways to carry out the method. Each block shown in FIG. 3 represents one or more processes, methods, or subroutines, carried out in the example method. Furthermore, the illustrated order of blocks is illustrative only and the order of the blocks can change. Additional blocks can be added or fewer blocks may be utilized or the order of the blocks may be changed, without departing from this disclosure. The method can begin at block 11.

At block 1, the plan generator 10 generates an original therapeutic plan according to a basic radiation dose.

When the radiation dose is too large during a formal radiation therapy, irreversible damage to the central nervous system, such as radiation encephalopathy, may happen after an incubation period. For example, edema and chromatin loosening may happen in the neuronal cells on the third day after the radiation therapy is completed, and obvious apoptosis can be observed on the seventh day. Moreover, the radiation doses applicable to different addicts 200 may be also different from each other (for example, the brain tissues of a juvenile are more sensitive to radiation). In addition, the occurrence of the radiation encephalopathy is also relative to other factors such as physical condition, hardening of the arteries, and immunity status of the body of the addict 200.

Therefore, the basic radiation dose should be determined before the formal radiation therapy. When radiation beams based on the basic radiation dose are directed to the target region 201 (including the nucleus accumbens of the brain), the release of dopamine in the nucleus accumbens is suppressed, thus the activities of the nucleus accumbens can be inhibited and reduced. Furthermore, the radiation beams based on the basic radiation dose do not cause death or permanent damage to the nucleus accumbens. Afterwards, the basic radiation dose can further be increased or decreased according to the individual situation, to obtain a target radiation dose applicable to the addict 200. As such, when the radiation beams are directed to the nucleus accumbens based on the target radiation dose, any unnecessary damage and side effect to the nucleus accumbens caused by an excessive radiation dose can be avoided. Ineffective treatment due to an insufficient radiation dose can also be avoided.

The basic radiation dose can be evaluated or determined according to experiences of doctors or existing medical data. Other factors, such as the type of tissue to be treated, the physical condition of the addict 200 (such as body density), may also be taken into consideration when determining the basic radiation dose. In at least one embodiment, the basic radiation dose can be input to the plan generator 10 by the doctors or other designated user through an input device (keyboard or mouse).

In at least one embodiment, the plan generator 10 determines original radiation data corresponding to the basic radiation dose, and generates the original therapeutic plan according to the determined original radiation data. The shape of the target region 201, activities of cells or tissues in the target region 201, may also be taken into consideration when determining the original radiation data.

At block 2, the therapeutic radiation device 20 receives the original therapeutic plan, and emits the radiation beams 24 to the target area 201 according to the original therapeutic plan. The radiation beams based on the basic radiation dose are adapted for lowering the activities of the nucleus accumbens.

Specifically, the therapeutic radiation device 20 emits the radiation beams 24 to the target area 201 according to the original therapeutic plan, the radiation dose of the radiation beams directed to the target region 201 is equal to the basic radiation dose.

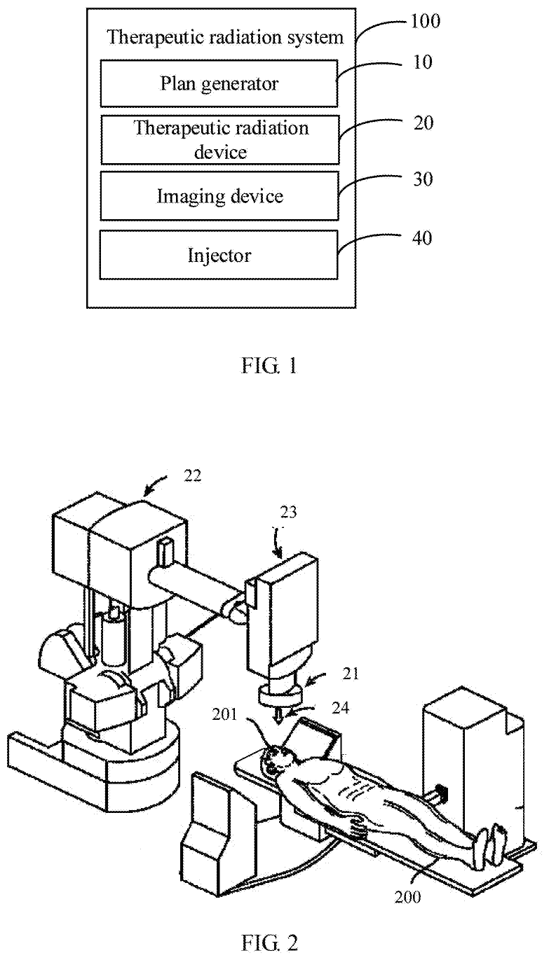

Referring to FIG. 2, the therapeutic radiation device 20 includes a radiation source 21, and emits the radiation beams 24 through the radiation source 21. In at least one embodiment, the therapeutic radiation device 20 further includes a driver 22 and a linear accelerator 23 fixed to the driver 22. The radiation source 21 is fixed to the linear accelerator 23. The driver 22 drives the radiation source 21, through the linear accelerator 23, to an appropriate position or orientation. The radiation source 21 emits the radiation beams 24 toward the target region 201 when the radiation source 21 is at the appropriate position. The driver 22 may be a robot. The therapeutic radiation device 20 can be a stereotactic radiosurgery (SRS) device or a stereotactic radiotherapy (SRT) device. The SRS device includes an X-knife, a gamma knife (Y knife), and a cyber knife (Cyber Knife). The SRT device includes a three-dimensional conformal radiation therapy (3DCRT) and a three-dimensional conformal intensity modulated radiation therapy (IMRT).

Furthermore, the radiation source 21 includes a bulb (not shown) that can generates the radiation beams 24. The original radiation data includes a tube current or a tube voltage of the bulb. The original radiation data may also include a radiation angle of the radiation beams 24, the number of times for radiation, and the duration of emitting radiation. The radiation source 21 can emit the radiation beams 24 to the target region 201 from different radiation angles.

In at least one embodiment, the system 100 further includes an injector 40. Since the treatment depends on the sensitivity of the nucleus accumbens to the radiation beams 24, a radiation sensitizer can be injected to the addict 200 through the injector 40, before the radiation source 21 emits the radiation beams 24. The radiation sensitizer can increase the sensitivity of the nucleus accumbens to the radiation beams 24. Therefore, with a same therapeutic effect, the radiation dose can be reduced when the radiation sensitizer is used. Thus, the radiation beams 24 can cause less damage to the nucleus accumbens and the survival rate of the cells of the nucleus accumbens cells can be improved. In at least one embodiment, the radiation sensitizer may include, but is not limited to, a DNA base analog, an electrophilic radiosensitizer (such as nitroimidazole compounds, nitroaromatic hydrocarbon compounds, and nitro heterocycle compounds), hypoxic cell radiosensitizers, bioreductive compounds, radiation damage repair inhibitors, sulfhydryl inhibitors, oxygen utilization inhibitors, oxygenates, cytotoxic sensitizers, targeted radiosensitizers, gene-related tumor radiosensitizers, and any combination thereof.

At block 3, the imaging device 30 captures images of the target area 201 to detect whether the nucleus accumbens recover activities in a preset duration after the original therapeutic plan is completed, and determines a target radiation dose by increasing or decreasing the basic radiation dose according to a detection of whether the nucleus accumbens recover activities.

In at least one embodiment, the evaluation device 30 may be a functional magnetic resonance imaging (fMRI) device. The fMRI device can measure changes in blood dynamics caused by neuronal activities, to determine whether the nucleus accumbens recover activities. The preset duration can be a theoretical duration in that the nucleus accumbens can recover activities after radiation. The preset duration can be determined by the experiences of the doctors or the radiotherapists. In at least one embodiment, the preset duration is about one week.

When the nucleus accumbens does not recover activities in the preset duration after the original therapeutic plan is completed, the basic radiation dose is indicated to be too high, and the radiation beams 24 based on the basic radiation dose are likely to cause permanent damage to the target region 201. On the other hand, when the nucleus accumbens recovers activities in the preset duration after the original therapeutic plan is completed, the radiation beams 24 based on the basic radiation dose are not likely to cause permanent damage to the target area 201, and the basic radiation dose can further be increased to obtain the target radiation dose that is applicable to the target area 201. That is, by increasing or decreasing the basic radiation dose based on the detection, the target radiation dose (an optimal radiation dose for the addict 200) can be obtained. When an actual radiation dose is greater than the target radiation dose, the radiation beams 24 based on the actual radiation dose may cause permanent damage to the nucleus accumbens. When the actual radiation dose is smaller than the target radiation dose, the treatment may be ineffective may be obtained although the actual radiation dose may also reduce the activities of the nucleus accumben. In this embodiment, the target radiation dose is less than 30 Gray (30 Gy).

At block 4, the plan generator 10 generates a target therapeutic plan according to the target radiation dose.

In at least one embodiment, the plan generator 10 divides the target radiation dose into a plurality of radiation sub-doses, and adjusts the original radiation data according to each radiation sub-dose to obtain the corresponding target radiation data. The plan generator 10 further generates the target therapeutic plan according to the target radiation data corresponding to each radiation sub-dose. Then, after the radiation beams 24 are emitted to the target region 201 according to the corresponding target radiation data, the total radiation dose of the radiation beams 24 directed to the target region 201 will be equal to the target radiation dose. Taking the target radiation dose as 5 Gy as an example, the plan generator 10 sets the radiation sub-dose as 0.05 Gy. After the radiation beams 24 are subsequently emitted toward the target region 201 according to the corresponding target radiation data, the total radiation dose reaches 5 Gy.

In another embodiment, the plan generator 10 may also adjust the original radiation data directly according to the target radiation dose to obtain the target radiation data, and generate the target therapeutic plan according to the target radiation data.

At block 5, the therapeutic radiation device 20 receives the target therapeutic plan, and emits the radiation beams 24 again to the target area 201 according to the target therapeutic plan.

In at least one embodiment, blocks 3 and 4 represent sub-processes beginning at block 41.

At block 41, the imaging device 30 captures images of the target area 201 to detect whether the nucleus accumbens recover activities in the preset duration after the original therapeutic plan is completed. If yes, the procedure goes to block 42; otherwise, the procedure goes to block 45.

At block 42, the plan generator 10 generates a first reference therapeutic plan according to a first reference radiation dose. The first reference radiation dose is determined by increasing the basic radiation dose.

At block 43, the therapeutic radiation device 20 receives the first reference therapeutic plan, and emits the radiation beams 24 again to the target area 201 according to the first reference therapeutic plan.

At block 44, the imaging device 30 captures images of the target area 201 to detect whether the nucleus accumbens recover activities in the preset duration after the first reference therapeutic plan is completed. If no, the procedure goes to block 45; otherwise, the procedure goes to block 46.

At block 45, the radiation beams 24 based on the first reference radiation dose is likely to cause permanent damage to the nucleus accumbens, and the imaging device 30 determines the target radiation dose to be equal to the basic radiation dose.

At block 46, the system 100 increases the first reference radiation dose, emits the radiation beams based on the increased first reference radiation dose, and evaluates the activities of the nucleus accumbens repeatedly until the nucleus accumbens do not recover activities under the radiation beams based on a current reference radiation dose, and determines the target radiation dose to be equal to a first reference radiation dose prior to the current reference radiation dose.

At block 47, the plan generator 10 generates a second reference therapeutic plan according to a second reference radiation dose. The second reference radiation dose is determined by decreasing the basic radiation dose.

At block 48, the therapeutic radiation device 20 receives the second reference therapeutic plan, and emits the radiation beams 201 again to the target area 201 according to the second reference therapeutic plan.

At block 49, the imaging device 30 captures images of the target area 201 to detect whether the nucleus accumbens recover activities in the preset duration after the second reference therapeutic plan is completed. If yes, the procedure goes to block 50; otherwise, the procedure goes to block 46.

At block 50, the second reference radiation dose is not likely to cause permanent damage to the nucleus accumbens, and the imaging device 30 determines the target radiation dose to be equal to the second reference radiation.

At block 51, the system 100 decreases the second reference radiation dose, emits the radiation beams based on the decreased second reference radiation dose, and evaluates the activities of the nucleus accumbens repeatedly until the nucleus accumbens recover activities under the radiation beams based on a current reference radiation dose, and determines the target radiation dose to be equal to the current reference radiation dose.

In actual use, the plan generator 10 and the therapeutic radiation device 20 can be independent from each other, and also can be combined into one single device.

By emitting radiation beams to the nucleus accumbens of the brain and controlling the radiation dose of the radiation beams, the activities of the nucleus accumbens is inhibited. This process is not lethal in relation to the nucleus accumbens, so that the addict can successfully quit the addiction without suffering irreversible damage.

The embodiments shown and described above are only examples. Therefore, many commonly-known features and details are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size, and arrangement of the parts within the principles of the present disclosure, up to and including the full extent established by the broad general meaning of the terms used in the claims. It will, therefore, be appreciated that the embodiments described above may be modified within the scope of the claims.

What is claimed is:

1. A therapeutic radiation system for treating addiction, comprising:
a plan generator configured to generate an original therapeutic plan according to a basic radiation dose;
a therapeutic radiation device configured to receive the original therapeutic plan, and emit radiation beams to a target area according to the original therapeutic plan, the target area comprising nucleus accumbens, the radiation beams based on the basic radiation dose being adapt for lowering activities of the nucleus accumbens; and
an imaging device configured to capture images of the target area to detect whether the nucleus accumbens recover activities in a preset duration after the original therapeutic plan is completed, and determine a target radiation dose by increasing or decreasing the basic radiation dose according to a detection of whether the nucleus accumbens recover activities.

2. The therapeutic radiation system of claim 1, wherein:
the plan generator is further configured to generate a target therapeutic plan according to the target radiation dose;
the therapeutic radiation device is further configured to receive the target therapeutic plan, and emit the radiation beams again to the target area according to the target therapeutic plan.

3. The therapeutic radiation system of claim 2, wherein:
the plan generator is further configured to generate a first reference therapeutic plan according to a first reference radiation dose when the nucleus accumbens recover activities in the preset duration after the original therapeutic plan is completed, the first reference radiation dose is determined by increasing the basic radiation dose;
the therapeutic radiation device is further configured to receive the first reference therapeutic plan, and emit the radiation beams to the target area according to the first reference therapeutic plan;
the imaging device is further configured to capture images of the target area to detect whether the nucleus accumbens recover activities in the preset duration after the first reference therapeutic plan is completed;
when the nucleus accumbens do not recover activities in the preset duration after the first reference therapeutic plan is completed, the imaging device determines the target radiation dose to be equal to the basic radiation dose; and
when the nucleus accumbens recover activities in the preset duration after the first reference therapeutic plan is completed, the therapeutic radiation system is further configured to increase the first reference radiation dose, emit the radiation beams based on the increased first reference radiation dose, and evaluate the activities of the nucleus accumbens repeatedly until the nucleus accumbens do not recover activities under the radiation beams based on a current reference radiation dose, and determine the imaging device determines the target radiation dose to be equal to a first reference radiation dose prior to the current reference radiation dose.

4. The therapeutic radiation system of claim 3, wherein:
the plan generator is further configured to generate a second reference therapeutic plan according to a second reference radiation dose when the nucleus accumbens do not recover activities in the preset duration after the original therapeutic plan is completed, the second reference radiation dose is determined by decreasing the basic radiation dose;
the therapeutic radiation device is further configured to receive the second reference therapeutic plan, and emit the radiation beams to the target area according to the second reference therapeutic plan;
the imaging device is further configured to capture images of the target area to detect whether the nucleus accumbens recover activities in the preset duration after the second therapeutic plan is completed;
when the nucleus accumbens recover activities in the preset duration after the second therapeutic plan is completed, the imaging device determines the target radiation dose to be equal to the second reference radiation dose, and
when the nucleus accumbens do not recover activities in the preset duration after the second therapeutic plan is completed, the therapeutic radiation system is configured to decrease the second reference radiation dose, emit the radiation beams based on the decreased second reference radiation dose, and evaluate the activities of the nucleus accumbens repeatedly until the nucleus accumbens recover activities under the radiation beams based on a current reference radiation dose, and determine the target radiation dose to be equal to the current reference radiation dose.

5. The therapeutic radiation system of claim 2, wherein the plan generator is further configured to determine original radiation data corresponding to the basic radiation dose, and generate the original therapeutic plan according to the determined original radiation data.

6. The therapeutic radiation system of claim 5, wherein the plan generator is configured to divide the target radiation dose into a plurality of radiation sub-doses, and adjust the original radiation data according to each of the plurality of radiation sub-doses to obtain corresponding target radiation data;
the plan generator is configured to generate the target therapeutic plan according to the target radiation data corresponding to each of the plurality of radiation sub-doses, wherein after the radiation beams are emitted to the target region according to the corresponding target radiation data, a total radiation dose of the radiation beams directed to the target region is equal to the target radiation dose.

7. The therapeutic radiation system of claim 6, wherein each of the original radiation data and the target radiation data comprises a tube current or a tube voltage of a bulb, a radiation angle of the radiation beams, a number of times for radiation, and a duration of emitting radiation.

8. The therapeutic radiation system of claim 1, wherein the preset duration is one week.

9. The therapeutic radiation system of claim 1, further comprising an injector configured to inject a radiation sensitizer to an addict before the therapeutic radiation device emits the radiation beams to the target region.

10. The therapeutic radiation system of claim 1, wherein the target radiation dose is less than 30 Gy.

11. A therapeutic radiation method for treating addiction, comprising:
generating an original therapeutic plan according to a basic radiation dose;
emitting radiation beams to a target area of an addict according to the original therapeutic plan, the target area comprising nucleus accumbens, the radiation beams based on the basic radiation dose being adapted for lowering activities of the nucleus accumbens; and
capturing images of the target area to detect whether the nucleus accumbens recover activities in a preset duration after the original therapeutic plan is completed, and determining a target radiation dose by increasing or decreasing the basic radiation dose according to a detection of whether the nucleus accumbens recover activities.

12. The therapeutic radiation method of claim 11, further comprising:
generating a target therapeutic plan according to the target radiation dose; and
emitting the radiation beams again to the target area according to the target therapeutic plan.

13. The therapeutic radiation method of claim 12, before generating the target therapeutic plan, the method further comprises:
generating a first reference therapeutic plan according to a first reference radiation dose when the nucleus accumbens recover activities in the preset duration after the original therapeutic plan is completed, the first reference radiation dose being determined by increasing the basic radiation dose;
emitting the radiation beams again to the target area according to the first reference therapeutic plan;
capturing images of the target area to detect whether the nucleus accumbens recover activities in the preset duration after the first reference therapeutic plan is completed;
when the nucleus accumbens do not recover activities in the preset duration after the first reference therapeutic plan, determining the target radiation dose to be equal to the basic radiation dose; and
when the nucleus accumbens recover activities in the preset duration after the first reference therapeutic plan is completed, increasing the first reference radiation dose, emitting the radiation beams based on the increased first reference radiation dose, and evaluating the activities of the nucleus accumbens repeatedly until the nucleus accumbens do not recover activities under the radiation beams based on a current reference radiation dose, and determining the imaging device determines the target radiation dose to be equal to a first reference radiation dose prior to the current reference radiation dose.

14. The therapeutic radiation method of claim 13, further comprising:
generating a second reference therapeutic plan according to a second reference radiation dose when the nucleus accumbens do not recover activities in the preset duration after the original therapeutic plan is completed, the second reference radiation dose being determined by decreasing the basic radiation dose;
emitting the radiation beams again to the target area according to the second reference therapeutic plan;
capturing images of the target area to detect whether the nucleus accumbens recover activities in the preset duration after the second therapeutic plan is completed;
when the nucleus accumbens recover activities in the preset duration after the second therapeutic plan is completed, determining the target radiation dose to be equal to the second reference radiation dose;
when the nucleus accumbens do not recover activities in the preset duration after the second therapeutic plan is completed, decreasing the second reference radiation dose, emitting the radiation beams based on the decreased second reference radiation dose, and evaluating the activities of the nucleus accumbens repeatedly until the nucleus accumbens recover activities under the radiation beams based on a current reference radiation dose, and determining the target radiation dose to be equal to the current reference radiation dose.

15. The therapeutic radiation method of claim 11, wherein generating the original therapeutic plan according to the basic radiation dose comprises:
determining original radiation data corresponding to the basic radiation dose; and
generating the original therapeutic plan according to the determined original radiation data.

16. The therapeutic radiation method of claim 15, wherein generating the target therapeutic plan according to the target radiation dose comprises:
dividing the target radiation dose into a plurality of radiation sub-doses;
adjusting the original radiation data according to each of the plurality of radiation sub-doses to obtain corresponding target radiation data; and
generating the target therapeutic plan according to the target radiation data corresponding to each of the plurality of radiation sub-doses, wherein after the radiation beams are emitted to the target region according to the corresponding target radiation data, a total radiation dose of the radiation beams directed to the target region is equal to the target radiation dose.

17. The therapeutic radiation method of claim 16, wherein each of the original radiation data and the target radiation data comprise a tube current or a tube voltage of a bulb, a radiation angle of the radiation beams, a number of times for radiation, and a duration of emitting radiation.

18. The therapeutic radiation method of claim 11, wherein the preset duration is one week.

19. The therapeutic radiation method of claim 11, further comprising:
injecting a radiation sensitizer to an addict before emitting the radiation beams to the target region.

20. The method of claim 11, wherein the target radiation dose is less than 30 Gy.
